(19) United States
(12) Patent Application Publication
(10) Pub. No.: US 2005/0261692 A1
(43) Pub. Date: Nov. 24, 2005

(54) Articulating tissue removal probe and methods of using the same

(75) Inventors: Harold F. Carrison, Pleasanton, CA (US); Leonard R. Novero, San Jose, CA (US); Nestor Aganon, San Jose, CA (US)

Correspondence Address: Bingham McCuthen, LLP, Suite 1800, Three Embarcadero, San Francisco, CA, 94111-4067, US

(73) Assignee: Scimed Life Systems, Inc., Maple Grove, MN (US)

(21) Appl. No.: 10/850,730

(22) Filed: May 21, 2004

(51) Int. Cl. A61B 17/32

(52) U.S. Cl. 606/79

(57) ABSTRACT

A medical probe comprises a probe shaft having proximal and distal shaft portions, and an operative element, such as a tissue removal element, associated with the distal shaft portion. The proximal and distal shaft portions can be positioned relative to each other in an axially aligned relationship and an axially non-aligned relationship at the interface between the shaft portions. For example, the ends of the shaft portions that engage each other can be beveled, in which case, relative rotation of the shaft portions will cause the angle between the portions to vary. Or, the shaft portions can hinge relative to each other to vary the angle between them. Thus, it can be appreciated that the probe can be introduced along a straight path via a small opening within the patient, and then the probe shaft portions can be moved relative to each other to reach off-axis tissue. The probe may have a drive shaft that extends within the probe shaft and on which the operative element is mounted. The drive shaft may have proximal and distal rigid shaft portions and a linkage between the drive shaft portions, so that the drive shaft can bend when the probe shaft portions are placed in their axially non-aligned relationship.

### FIELD OF THE INVENTION

The field of the invention pertains to medical devices and methods for removing tissue, and in particular, vertebral bone and intervertebral disc tissue.

### BACKGROUND OF THE INVENTION

The spinal column consists of thirty-three bones called vertebra, the first twenty-four vertebrae of which make up the cervical, thoracic, and lumbar regions of the spine and are separated from each other by “pads” of tough cartilage called “intervertebral discs,” which act as shock absorbers that provide flexibility, stability, and pain-free movement of the spine.

FIGS. 1 and 2 illustrate a portion of a healthy and normal spine, and specifically, two vertebra 10 and two intervertebral discs 12 (only one shown). The posterior of the vertebra 10 includes right and left transverse processes 14R, 14L, right and left superior articular processes 16R, 16L, and a spinous process 18. Muscles and ligaments that move and stabilize the vertebra 10 are connected to these structures. The vertebra 10 further includes a centrally located lamina 20 with right and left lamina 20R, 20L, that lie in between the spinous process 18 and the superior articular processes 16R, 16L. Right and left pedicles 22R, 22L are positioned anterior to the right and left transverse processes 14R, 14L, respectively. A vertebral arch 24 extends between the pedicles 22 and through the lamina 20. The anterior of the vertebra 10 includes a vertebral body 26, which joins the vertebral arch 24 at the pedicles 22. The vertebral body 26 includes an interior volume of reticulated, cancellous bone (not shown) enclosed by a compact cortical bone 30 around the exterior. The vertebral arch 24 and vertebral body 26 make up the spinal canal (i.e., the vertebral foramen 32), which is the opening through which the spinal cord 34 and epidural veins (not shown) pass. Nerve roots 36 laterally pass from the spinal cord 34 out through canals 38 in the side of the spinal column formed between the pedicles 22. Structurally, the intervertebral disc 12 consists of two parts: an inner gel-like nucleus (nucleus pulposis) 40 located at the center of the disc 12, and tough fibrus outer annulus (annulus fibrosis) 42 surrounding the nucleus 40.

A person may develop any one of a variety of debilitating spinal conditions and diseases. For example, as illustrated in FIG. 3, when the outer wall of the disc 12′ (i.e., the annulus fibrosis 42) becomes weakened through age or injury, it may tear allowing the soft inner part of the disc 12 (i.e., the nucleus pulposis 40) to bulge out, forming a hernia 46. The herniated disc 12′ often pinches or compresses the adjacent dorsal root 36 against a portion of the vertebra 10, resulting in weakness, tingling, numbness, or pain in the back, legs or arm areas.

Often, inflammation from disc herniation can be treated successfully by nonsurgical means, such as rest, therapeutic exercise, oral anti-inflammatory medications or epidural injection of corticosterioids. In some cases, however, the disc tissue is irreparably damaged, in which case, surgery is the best option.

Besides disc hernias, other debilitating spinal conditions or diseases may occur. For example, spinal stenosis, which results from new bone and soft tissue growth on a vertebra, reduces the space within the spinal canal. When the nerve roots are pinched, a painful, burning, tingling, and/or numbing sensation is felt down the lower back, down legs, and sometimes in the feet. As illustrated in FIG. 2, the spinal canal 32 has a rounded triangular shape that holds the spinal cord 34 without pinching. The nerve roots 36 leave the spinal canal 32 through the nerve root canals 38, which should be free of obstruction. As shown in FIG. 4, new bone growth 48 (e.g., bone spurs) within the spinal canal 32, and specifically from the diseased lamina 20, causes compression of the nerve roots, which leads to the pain of spinal stenosis. Spinal stenosis may be treated by performing a laminectomy in order to relieve pressure on the nerve root 36 impinged by the bone growth 48. Along with the laminectomy, a foraminotomy, (i.e., enlarging of the channel from which the nerve roots 36 exit is performed). Depending on the extent of the bone growth, the entire lamina and spinal process may be removed.

Another debilitating bone condition is a vertebral body compression fracture (VCF), which may be caused by spinal injuries, bone diseases such as osteoporosis, vertebral hemangiomas, multiple myeloma, necorotic lesions (Kummel's Disease, Avascular Necrosis), and metastatic disease, or other conditions can cause painful collapse of vertebral bodies. VCFs are common in patients who suffer from these medical conditions, often resulting in pain, compromises to activities of daily living, and even prolonged disability.

On some occasions, VCFs may be repaired by cutting and removing damaged bone tissue inside a vertebra to create a void, and then injecting a bone cement percutaneously into the void. This is typically accomplished percutaneously through a cannula to minimize tissue trauma. The hardening (polymerization) of the cement media serves to buttress the bony vault of the vertebral body, providing both increased structural integrity and decreased pain associated with micromotion and progressive collapse of the vertebrae.

Thus, it can be appreciated that in many spinal treatment procedures, bone and/or disc tissue must be removed in order to release pressure from neural tissue or rebuild the vertebra. In order to access a target site, a physician can insert an access cannula through a patient's skin to reach target bone and/or disc tissue to be removed. A tissue removal probe can then be inserted through the cannula and be used to remove target tissue, such as the gel-like nuclear tissue within the intervertebral disc or the cancellous bone tissue within the vertebral body. Notably, such tissue removal probe is laterally constrained within the cannula (or if a cannula is not provided, constrained by the many layers of tissue that the device must traverse to reach the target tissue), and thus, can only be effectively moved along its longitudinal axis, thereby limiting the amount of tissue that can be removed to the tissue that is on-axis. As such, the tissue removal probe may have to be introduced through several access points within the anatomical body (e.g., the disc or vertebral body) that contains the target tissue in order to remove the desired amount of the tissue. As can be appreciated, such technique increases the time of the spinal procedure as well as surgical risk.

Tissue removal probes having steering capability have been used to overcome the above described problem. Such tissue removal probes generally have a steering wire secured to a distal end of the probe shaft. The steering wire can be tensioned during use, which in turn, causes the distal end of the probe to bend. By allowing the tissue removal probe to bend while the probe is laterally constrained within the access cannula (or the layers of tissue if a cannula is not provided), the distal end of the tissue removal probe can be steered to reach target tissue that cannot be normally reached by tissue removal probes having a straight configuration. However, use of a steering wire to bend a tissue removal probe may not provide sufficient rigidity for the probe to maintain its bent shape during use. For example, during use, surrounding tissue at a target site may exert a force on the tissue removal probe, which causes the probe to unbent itself. This in turn limits the range of target area which the tissue removal probe can reach.

A rigid tissue removal probe can be provided with a bend distal end, so that off-axis tissue can be reached. The bend distal probe end, however, increases the profile of the probe, thereby requiring the access opening through which the probe is introduced into the patient to be increased, thereby increasing patient discomfort and recovery time. In addition, the curvature of the bent distal end is fixed, thereby limiting access to the off-axis tissue.

There, thus, remains a need to provide for improved tissue removal device and methods for use during spinal treatment and other surgeries.

### SUMMARY OF THE INVENTION

In accordance with the present inventions, a medical probe is provided. The medical probe comprises a probe shaft having proximal and distal shaft portions that can move relative to each other, and an operative element, such as a tissue removal element, associated with the distal shaft portion. In one embodiment, the proximal and distal shaft portions are rigid and straight to facilitate percutaneous introduction of the probe into the patient, but may be semi-rigid or flexible and/or curved as well. The medical probe may optionally have a drive shaft disposed within the probe shaft, in which case, the operative element may be mounted to the drive shaft. The operative element may be variously associated with the distal shaft portion. For example, if the operative element is a tissue removal element, the distal shaft portion may include a window through which the tissue removal element is exposed. Or the tissue removal element may extend distally of the distal shaft portion. In one embodiment, the operative element, as a tissue removal element, is rotatable, but alternatively, may move in other directions, e.g., longitudinally, in order to remove tissue. The medical probe may optionally have an adapter configured to mate with a drive unit.

In accordance with a first aspect of the present inventions, the proximal and distal shaft portions can be positioned relative to each other between axially aligned and axially non-aligned relationships. The shaft portions may be, e.g., rotatably or hingedly coupled to each other.

In accordance with a second aspect of the present inventions, the proximal shaft portion has a first beveled end, and the distal shaft portion has a second beveled end rotatably engaged with the beveled end. In this manner, the respective beveled ends interact with each other, such that an angle formed between the shaft portions can be varied when the shaft portions are rotated relative to each other. In one embodiment, the beveled ends are beveled at the same angle, so that the proximal and distal shaft portions can be placed in an axially aligned relationship. The medical probe may optionally comprise a rod rotatably disposed through the proximal shaft portion and fixedly coupled to the distal shaft portion. In this manner, the distal shaft portion can be rotated relative to the proximal shaft portion by rotating the rod. In this case, the medical probe may optionally comprise a deformable connector coupled between the rod and the distal shaft portion adjacent an interface between the proximal and distal shaft portions. In this manner, stress between the rod and distal shaft portion can be minimized.

In accordance with a third aspect of the present inventions, the medical probe comprises a hinge coupled between the proximal and distal shaft portions, such that an angle formed between the shaft portions can be varied when the shaft portions are hinged relative to each other. The medical probe may optionally comprise at least one pull wire disposed through the proximal shaft portion and fixedly coupled to the distal shaft portion. In this manner, the distal shaft portion can be hinged relative to the proximal shaft portion by pulling the pull wire(s). In one embodiment, the hinge comprises a pin mounted to the distal shaft portion, in which case, a pair of pull wires can be counterwound around the pin. In this manner, the distal shaft portion can be hinged relative to the proximal shaft portions in opposite directions by alternately pulling on the pull wires.

In accordance with a fourth aspect of the present inventions, a method of performing a medical procedure is performed on a patient. The method comprises introducing the probe into the patient while the proximal and distal probe portions are in an axially aligned relationship. The method further comprises placing the proximal and distal shaft portions in an axially non-aligned relationship, and then operating the operative element. Thus, it can be appreciated that the probe can be introduced along a straight path via a small opening within the patient, and then articulated to reach tissue that is off-axis from the path. In one preferred method, the operative element is operated after the distal shaft portion has been rotated relative to the proximal shaft portion. If the medical procedure involves removing tissue, such as bone tissue or intervertebral disc tissue, the operative element, as the tissue removal element, can be rotated to remove the tissue.

In accordance with a fifth aspect of the present inventions, the proximal and distal shaft portions are configured to rotate relative to each other, and the medical probe further comprises a rod disposed through the proximal shaft portion and fixedly coupled to the distal shaft portion, and an actuator mounted to the proximal portion in an axially sliding relationship. The rod comprises an obliquely extending slot, and the actuator comprising a pin slidably engaged within the slot. In this manner, axial movement of the actuator rotates the distal shaft portion via the rod. In one embodiment, reciprocatable axial movement of the actuator may rotate the distal shaft portion relative to the proximal shaft portion. The proximal and distal shaft portions may be rigid, but alternatively may be semi-rigid or flexible. If a drive shaft is provided, it can extend through the rod.

In accordance with a sixth aspect of the present inventions, the medical probe further comprises a drive shaft rotatably disposed within the probe shaft, in which case, the operative element will be mounted to the drive shaft. The drive shaft has a proximal rigid shaft portion associated with the proximal probe shaft portion, a distal rigid shaft portion associated with the distal probe shaft portion, and a linkage (e.g., a bellow, coil, U-joint, or beveled gear set) coupling the proximal and distal drive shaft portions. In this manner, the drive shaft may bend at the interface between the proximal and distal probe shaft portions without undergoing excessive stress.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention. It should be noted that the figures are not drawn to scale and that elements of similar structures or functions are represented by like reference numerals throughout the figures. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a perspective view of a portion of a spine;

FIG. 2 is a top view of a vertebra with a healthy intervertebral disk;

FIG. 3 is a top view of a vertebra with a herniated intervertebral disk;

FIG. 4 is a top view of a vertebral with spinal stenosis;

FIG. 5A is a side cross sectional view of a tissue removal device in accordance with some embodiments of the invention;

FIG. 5B is a side cross sectional view of the tissue removal device of FIG. 5A, showing a distal portion of the device rotated relative to a proximal portion;

FIG. 6A is a side cross sectional view of a tissue removal device in accordance with other embodiments of the invention, showing the device having a connector connecting a proximal portion to a distal portion of the device;

FIG. 6B is a side cross sectional view of the tissue removal device of FIG. 6A, showing a distal portion of the device rotated relative to a proximal portion;

FIG. 7A is a perspective view of a tissue removal device in accordance with other embodiments of the invention, showing the device having a wire coupled to a rotatable connection;

FIG. 7B is a perspective view of the tissue removal device of FIG. 7A, showing a distal portion of the device rotated relative to a proximal portion;

FIGS. 8A and 8B illustrate a drive shaft for a tissue removal element in accordance with some embodiments of the invention, showing the drive shaft having a bellow;

FIGS. 9A and 9B illustrate a drive shaft for a tissue removal element in accordance with other embodiments of the invention, showing the drive shaft having a spring;

FIGS. 10A and 10B illustrate a drive shaft for a tissue removal element in accordance with other embodiments of the invention, showing the drive shaft having a U-joint;

FIGS. 11A and 11B illustrate a drive shaft for a tissue removal element in accordance with other embodiments of the invention; showing the drive shaft having a bevel gear;

FIG. 12 illustrates a variation of a distal portion of a sheath that can be used with embodiments of the invention;

FIG. 13 illustrates a tissue removal element in a form of a cutting basket that can be used with embodiments of the invention;

FIG. 14 illustrates a tissue removal element in a form of a drill bit that can be used with embodiments of the invention;

FIG. 15 illustrates a tissue removal device in accordance with other embodiments of the invention, showing the tissue removal device having a switch for positioning a distal portion of the device; and

FIGS. 16A-16C are perspective views showing a method of using the tissue removal device of FIG. 5A to remove tissue within a herniated intervertebral disc.

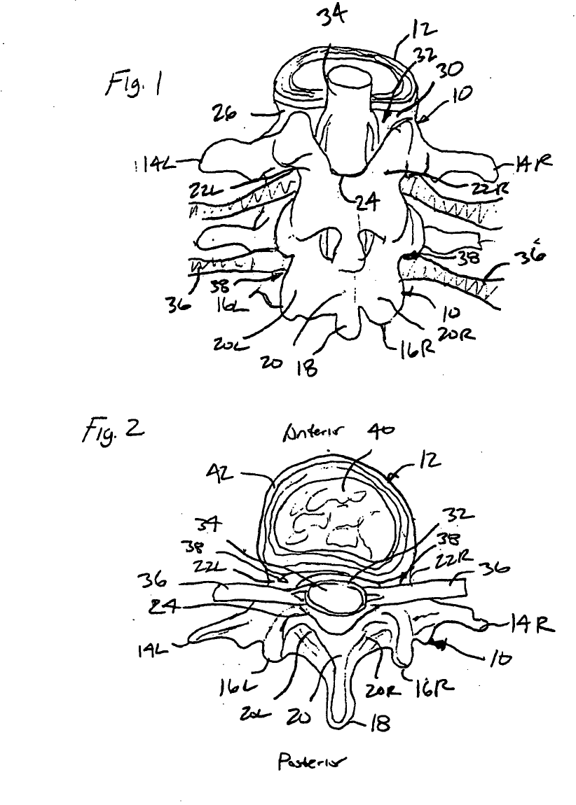

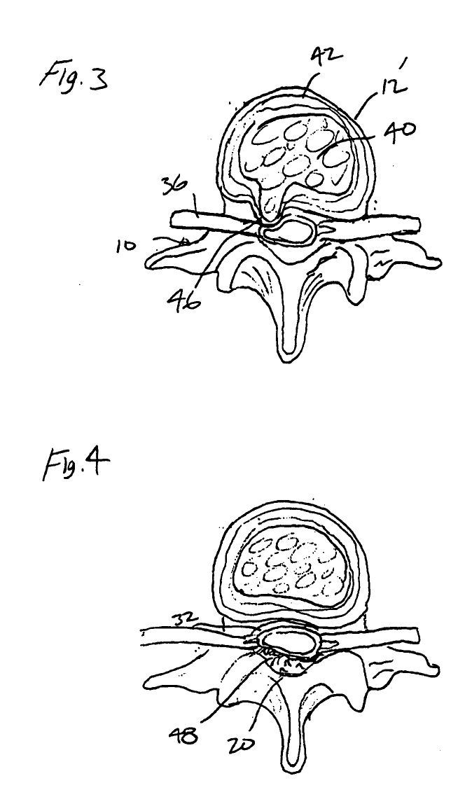

### DETAILED DESCRIPTION OF THE EMBODIMENTS

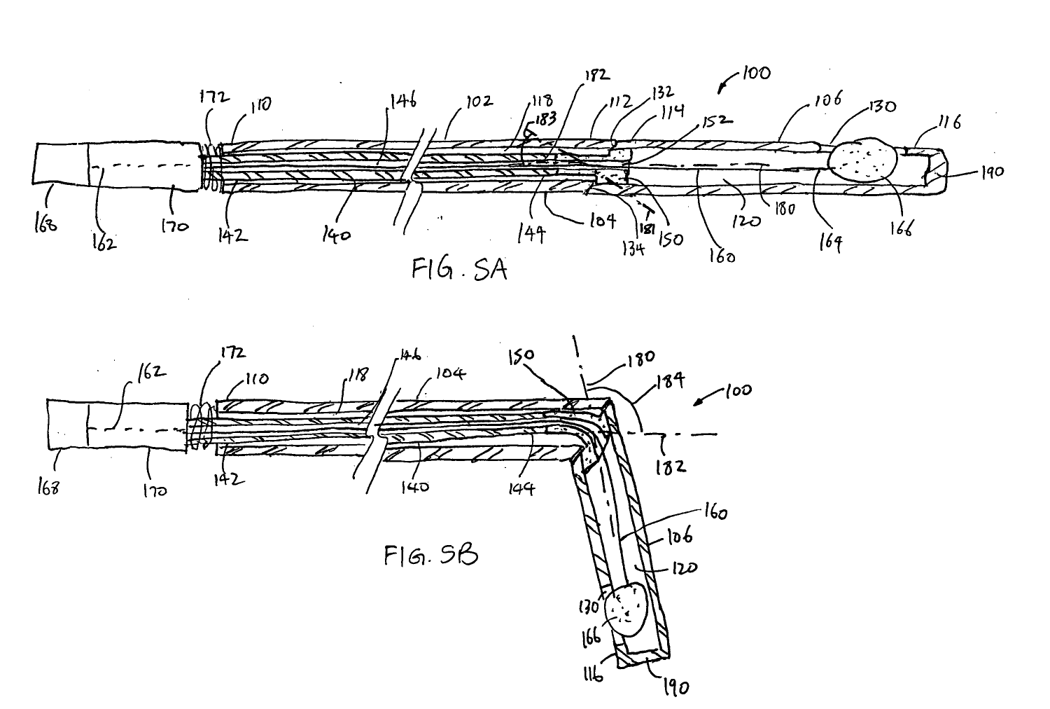

FIGS. 5A and 5B illustrate a tissue removal probe 100 constructed in accordance with one preferred embodiment of the present invention. The probe 100 includes a probe shaft 102 having a proximal probe shaft portion 104 that extends along a longitudinal axis 182, and a distal probe shaft portion 106 that extends along a longitudinal axis 180. In the illustrated embodiments, the proximal and the distal portions 104, 106 each has a longitudinal profile that is substantially rectilinear. Alternatively, either or both of the proximal and the distal portions 104, 106 can have a curvilinear or a bent configuration. The probe shaft portions 102 and 104 are rotatably coupled together at an interface. The proximal portion 104 of the probe shaft 102 includes a proximal end 110, a distal beveled end 112, and a lumen 118 extending between the proximal and distal ends 110, 112. The distal portion 106 of the probe shaft 102 includes a proximal beveled end 114, a distal end 116, and a lumen 120 extending between the proximal and distal ends 114, 116.

The beveled end 112 of the proximal probe shaft portion 102 and the beveled end 114 of the distal probe shaft portion 102 engage in a manner that allows the shafts portions 104, 106 to be placed in an axially aligned relationship at the interface (i.e., the longitudinal axes 182, 180 are coextensive with each other at the interface) (see FIG. 5A), and an axially non-aligned relationship (i.e., the longitudinal axes 182, 180 form a non-0 or non-180 degree angle 184) (see FIG. 5B). In particular, the ends 112, 114 are beveled at the same angle (at angle 183 formed between the longitudinal axis 182 and a longitudinal axis 181 extending perpendicularly to the surfaces of the beveled ends 112, 114), such that, given a particular rotation of one shaft portions 104, 106 relative to each other, the longitudinal axes 182, 180 of the probe shaft portions 104, 106 are coextensive. In the illustrated embodiment, the angle 183 is 45 degrees, although other values for the angle 183 may be used. When one of the shaft portions 104, 106 is rotated about its respective longitudinal axis 182, 180 relative to the other shaft portion 104, 106, however, the longitudinal axes 182, 180 become axially non-aligned, thereby forming an angle 184 between the shaft portions 104, 106. This angle 184 can be varied by further rotating the shaft portions 104, 106 relative to each other.

In the illustrated embodiment, the probe 100 has an actuator for rotating the distal shaft portion 106 relative to the proximal shaft portion 104. In particular, the probe 100 includes a rod 140 disposed coaxially within the lumen 118 of the proximal shaft portion 104. The rod 140 includes a proximal end 142 secured to a handle 170, a distal end 144 coupled to the proximal end 114 of the distal portion 106 of the probe shaft 102, and a lumen 146 extending between the proximal and distal ends 142, 144. During use, the handle 170 can be torqued to rotate the rod 140, which causes the distal shaft portion 106 to rotate relative to the proximal portion 104 of the probe shaft 102. The handle 170 is preferably composed of a durable and rigid material, such as medical grade plastic, and is ergonomically molded to allow a physician to more easily manipulate the distal shaft portion 106. Thus, rotation of the handle 170 causes the rod 140, and thus the distal shaft portion 106, to rotate relative to the proximal shaft portion 104.

In the illustrated embodiment, the distal end 144 of the rod 140 is secured to the distal shaft portion 106 by a connector 150. The connector 150 includes a lumen 152 for housing a drive shaft 160. The connector 150 is made from an elastic material, such as plastic, rubber, aluminum, or other metals or alloys, such that the connector 150 can undergo deformation as the distal shaft portion 106 is being rotated relative to the proximal portion 104 of the probe shaft 102. In other embodiments, the connector 150 can be a spring, or have other configurations. A compression spring 172 is disposed between the proximal end 110 of the proximal shaft portion 104 and the handle 170, and is configured to exert a force that tends to separate the handle 170 axially away from the proximal end 110. Such feature allows the compression spring 172 to pull the rod 140 proximally relative to the proximal shaft portion 104, thereby ensuring that the proximal end 114 of the distal shaft portion 106 maintains contact with the distal end 112 of the proximal shaft portion 104 as the distal shaft portion 106 is being rotated relative to the proximal shaft portion 104. In other embodiments, instead of using the compression spring 172, the probe 100 can include other devices, mechanisms, or materials that pull the rod 140 proximally relative to the proximal shaft portion 104. Also, in other embodiments, the probe 100 does not include the compression spring 172. In such cases, an operator of the probe 100 can pull the handle 170 relative to the proximal shaft portion 104 of the sheath 102 to keep the proximal tip 132 in contact with the distal tip 134 during use.

The drive shaft 160 is disposed coaxially within the lumen 146 of the rod 140. The drive shaft 160 has a proximal end 162 secured to a driver 168, and a distal end 164 secured to a tissue removal element 166. The driver 168 may take the form of a standard rotary drive used for powering medical cutting instruments. In the illustrated embodiments, the driver 168 is secured to a proximal end of the handle 170. In alternative embodiments, the driver 168 can be secured to other locations on the handle 170, or can be a separate unit from the handle 170. During use, the driver 168 is activated to rotate the drive shaft 160, which in turn, causes the tissue removal element 166 to rotate. The tissue removal element 166 extends at least partially out of an opening 130 (a cutting window) located at a side wall of the distal shaft portion 106. The cutting window 130 exposes a portion of the tissue removal element 166, such that the tissue removal element 166 cuts and abrades tissue only on one lateral side (top) of the tissue removal probe 100, while protecting tissue at the opposite lateral side (bottom) of the tissue removal probe 100. In the illustrated embodiments, the cutting window 130 has a rectangular shape, but can have other shapes as well. In the illustrated embodiments, drive shaft 160 is made of a flexible material, such as coiled or braided stainless steel. In other embodiments, the drive shaft 160 can be made from other materials. In the illustrated embodiments, the distal end of the drive shaft 160 extends to the tissue removal element 166. Alternatively, the distal end of the drive shaft 160 extends through the tissue removal element, and is rotatably secured to a wall 190 at the distal end 116 of the distal shaft portion 106.

In some embodiments, the drive shaft 160 can be made slidable relative to the distal shaft portion 106, thereby allowing the tissue removal element 166 to be positioned axially relative to and within the cutting window 130. As can be appreciated, longitudinal movement of the drive shaft 160 slides the tissue removal element 166 along the cutting window 130 between a proximal position and a distal position. As such, the cutting window 130 advantageously limits the tissue removed to that which extends along the cutting window 130. At the same time, the length of the cutting window 130 allows a length of tissue to be removed without having to move the probe shaft 102. The length of the cutting window 130 will depend upon the length of the tissue that is to be removed. In the illustrated embodiment, the length of the cutting window 130 is in the range of 0.25-1.5 inches.

To facilitate placement and maintenance of the cutting window 130 at the tissue removal site, the distal and proximal portions 106, 104 of the probe shaft 102 are preferably rigid (e.g., it can be composed of a rigid material, or reinforced with a coating or a coil to control the amount of flexing), so that the probe shaft 102 provides a more stable platform from which to remove tissue. The probe shaft 102 can be made from a variety of materials, such as polymers, plastics, stainless steel, aluminum, or other metals or alloys. The materials used in constructing the probe shaft 102 may also comprise any of a wide variety of biocompatible materials. In some embodiments, a radiopaque material, such as metal (e.g., stainless steel, titanium alloys, or cobalt alloys) or a polymer (e.g., ultra high molecular weight polyethylene) may be used, as is well known in the art. In the illustrated embodiments, the probe shaft 102 has a cross sectional shape that is circular. Alternatively, the probe shaft 102 can have other cross sectional shapes. The outer cross sectional dimension of the probe shaft 102 is preferably less than ½ inch, but other dimensions for the outer cross sectional dimension of the probe shaft 102 may also be appropriate, depending on the particular application or clinical procedure. The lumen 118 of the proximal portion of the probe shaft 102 should have a cross sectional dimension so as to allow the rod 140 to be rotatably housed therein.

In the illustrated embodiments, the tissue removal element 166 is a burr that includes abrasive particles, such as diamond dust, disposed on a surface of the burr. In other embodiments, instead of, or in addition to, having diamond dust, parts of the surface of the burr can be removed to create an abrasive surface. The burr can also include one or more grooves formed along the surface of the burr. In such case, the groove(s) allows bone particles that have been removed to travel proximally and away from a target site. The burr is preferably made from a tough material, such as steel or other alloys, so that it could penetrate or cut into bone tissue without being damaged. In the illustrated embodiments, the tissue removal element 166 has an elliptical profile. Alternatively, the tissue removal element 166 can have other shapes, such as a spherical shape or a cylindrical shape.

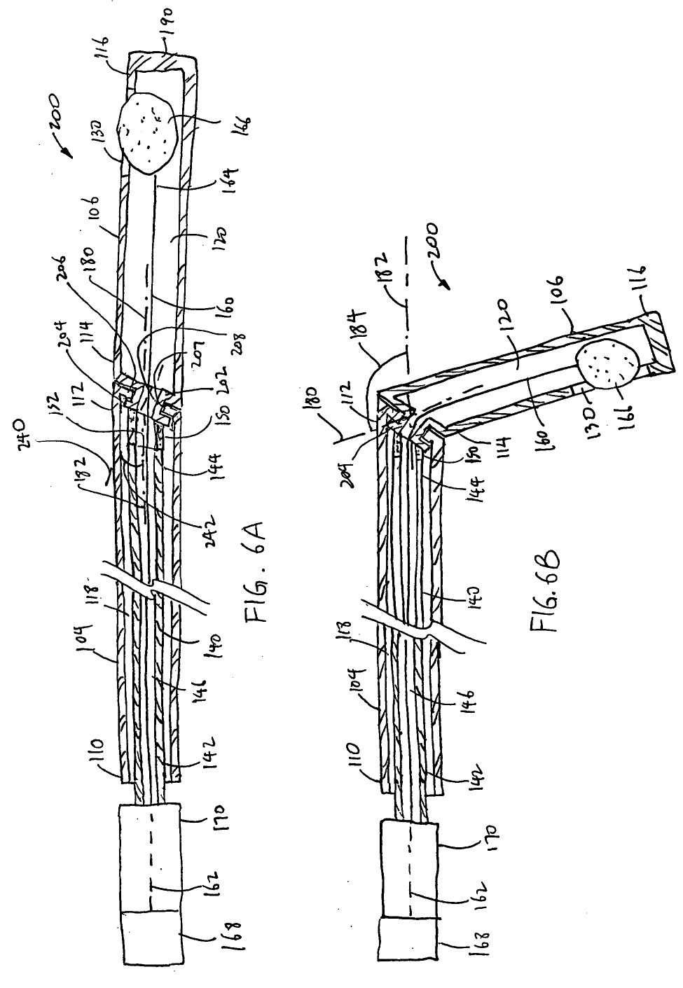

FIGS. 6A and 6B illustrate a tissue removal probe 200 constructed in accordance with another embodiment of the invention. The probe 200 is similar to the probe 100, with the exception that the probe 200 does not include the compression spring 172, and the proximal end 114 of the distal probe shaft portion 106 is rotatably coupled to the distal end 112 of the proximal probe shaft portion 104 via a connector 202. The connector 202 is secured to the distal portion 106 of the probe shaft 102, and is configured to guide the distal shaft portion 106 as it rotates relative to the proximal shaft portion 104.

In the illustrated embodiment, the distal end 112 of the proximal shaft portion 104 includes a first flange 206 defining a circular opening 207. The connector 202 includes a shaft 208 that extends through the circular opening 207, and a second flange 204 secured to the shaft 208. The flanges 204, 206 secure the distal shaft portion 106 to the proximal portion 104 of the probe shaft 102, and prevents the distal shaft portion 106 from separating from the proximal shaft portion 104 as the distal shaft portion 106 is being rotated relative to the proximal shaft portion 104. In this embodiment, the compression spring 172 is not necessary because the connector 202 functions to keep the distal and the proximal portions 106, 104 of the probe shaft 102 in contact during use. Alternatively, the probe 200 can include the compression spring 172 for maintaining the connector 150 in tension during use. In the illustrated embodiment, the connector 150 secures the distal end 144 of the rod 140 to the connector 202. The connector 150 undergoes deformation as the distal shaft portion 106 is being rotated relative to the proximal shaft portion 104.

In other embodiments, instead of having the connector 202 be associated with the distal shaft portion 106, the connector 202 can be associated with the proximal portion 104 of the probe shaft 102. In such cases, the proximal end 114 of the distal shaft portion 106 includes a first flange defining a circular opening, and the distal end 112 of the proximal shaft portion 104 includes the connector 202. It should be noted that the connector 202 is not limited to the configuration illustrated previously, and that the connector 202 can have other configurations in alternative embodiments.

In the above described embodiments, the distal probe shaft portion 104 is rotatably coupled to the proximal probe shaft portion 102. That is, the interface between the respective shaft portions 102, 104 allows the distal probe shaft portion 104 to rotate about or around the longitudinal axis 182 of the proximal probe shaft portion 102. The probe shaft portions, however, can be coupled in other manners in order to alternately place them in axially aligned and non-aligned relationships.

For example, FIGS. 7A and 7B illustrate a tissue removal probe 300 constructed in accordance with other embodiments of the invention. The probe 300 is similar to the probe 100, except that the distal probe shaft portion 106 is hingedly coupled to the proximal probe shaft portion 104. That is, the distal shaft portion 106 rotates about an axis 301 perpendicular to the longitudinal axis 182. In the illustrated embodiment, the probe 300 includes a hinge pin 302 that couples the distal shaft portion 106 to the proximal shaft portion 104 in a hinged configuration. In the illustrated embodiment, the hinge pin 302 is fixedly secured to the distal shaft portion 106, and is rotatable relative to the proximal shaft portion 104.

The probe 300 further includes an actuator for rotating the distal shaft portion 104 relative to the proximal shaft portion 102. In particular, the probe 300 includes a first and second wires 310, 312 that are counterwound around the hinge pin 302. That is, the first wire 310 wraps around the circumference of the hinge pin 302 in a first direction, and the second wire 312 wraps around the circumference of the hinge pin 302 in a second opposite direction. The distal tips (not shown) of the wires 310, 312 are secured to the hinge pin 302 using a suitable means, such as welding or soldering. During use, either of the wires 310, 312 can be selectively pulled to rotate the hinge pin 302, thereby causing the distal shaft portion 106 to hinge relative to the proximal shaft portion 104. For example, the first wire 310 can be pulled in a direction 320 to rotate the hinge pin 302 in a first direction 322. The rotation of the hinge pin 302, in turn, hinges the distal shaft portion 106 relative to the proximal shaft portion 104 in a direction, as indicated by arrow 324, to place the proximal and distal shaft portions 104, 106 from their axially aligned relationship (FIG. 7A) to their axially non-aligned relationship (FIG. 7B). The second wire 312 can be pulled in the direction 320 to rotate the hinge pin 302 in the opposite direction. The rotation of the hinge pin 302, in turn, hinges the distal shaft portion 106 relative to the proximal shaft portion 104 in the opposite direction to place the proximal and distal shaft portions 104, 106 from their axially non-aligned relationship (FIG. 7B) to their axially aligned relationship (FIG. 7A).

It should be appreciated that providing a probe shaft having rigid distal and proximal portions that are rotatably coupled prevents or at least reduces the risk of the tissue removal probe unbending itself, thereby allowing the tissue removal probe to substantially maintain its bent shape during use. Although several embodiments of a tissue removal probe have been described, it should be noted that the tissue removal probe should not be limited to the configurations described previously, and that the tissue removal probe can have other configurations in alternative embodiments as long as a distal portion of the probe can be rotated relative to a proximal portion to form a bent profile during use.

Also, in other embodiments, any of the embodiments of the tissue removal probe described herein can optionally have irrigation and/or aspiration capability. For example, the tissue removal probe 100 can include an irrigation tube and/or an aspiration tube disposed in the lumen 146 of the rod 140. The irrigation tube terminates at an irrigation outlet port in the distal end 116 and proximally terminates at an irrigation inlet port in a proximal adapter. Likewise, the aspiration tube terminates at an aspiration entry port in the distal end 116 and proximally terminates at an aspiration outlet port in the proximal adapter. As can be appreciated, a pump (not shown) can be connected to the irrigation inlet port on the proximal adapter in order to flush irrigation fluid, such as saline, through the irrigation tube and out the irrigation outlet port. The irrigation fluid helps cool the drive shaft and/or the tissue removal element, while the tissue removal element is rotating at high speed and grinding against tissue. The media also washes away debris at the target site and tissue removal element. A vacuum (not shown) can be connected to the aspiration outlet port on the proximal adapter in order to aspirate the removed tissue into the aspiration inlet port, through the aspiration tube, and out of the aspiration outlet port. Because there are separate irrigation and aspiration tubes, both the pump and aspirator can be activated simultaneously or separately.

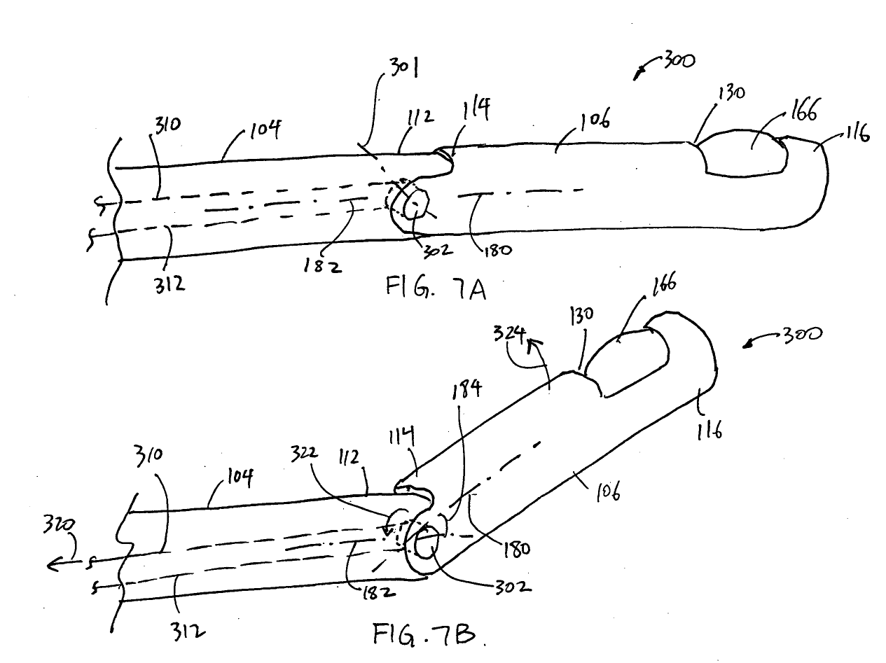

In the embodiments described previously, the drive shaft 160 is flexible along its entire length, such that the drive shaft 160 can be bent along with the probe as a distal portion of the tube is rotated to form a bent configuration with a proximal portion of the tube. In alternative embodiments, the drive shaft 160 can have other configurations. For example, FIGS. 8A and 8B illustrate a drive shaft 500 that can be used with any of the embodiments of the tissue removal probe described herein. The drive shaft 500 includes a proximal portion 502, a distal portion 504, and a bellow 506 connected between the proximal and the distal portions 502, 504. The proximal and the distal portions 502, 504 can be made from a relatively stiff materials such that the proximal and the distal portions 502, 504 remain substantially unbent during use. In such cases, a bending of the drive shaft 500 takes place at the bellow 506, which allows the distal portion 504 to bent relative to the proximal portion 502. The bellow 506 can transmit torqueing force to rotate the tissue removal element 166 even when the drive shaft 500 is bent.

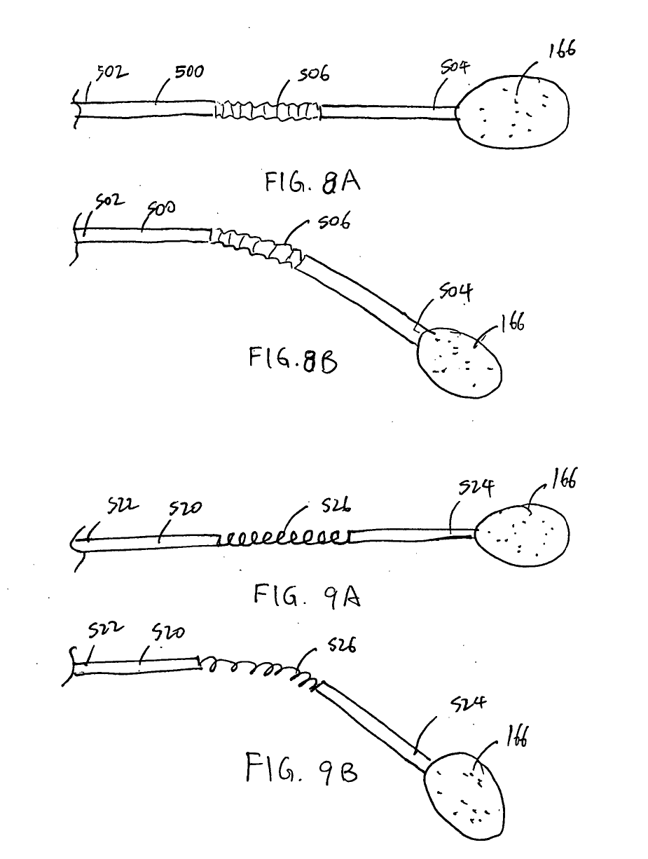

FIGS. 9A and 9B illustrate another drive shaft 520 that can be used with any of the embodiments of the tissue removal probe described herein. The drive shaft 520 includes a proximal portion 522, a distal portion 524, and a spring 526 connected between the proximal and the distal portions 522, 524. The proximal and the distal portions 522, 524 can be made from a relatively stiff materials such that the proximal and the distal portions 522, 524 remain substantially unbent during use. In such cases, a bending of the drive shaft 520 takes place at a coil 526, which allows the distal portion 524 to bent relative to the proximal portion 522. The coil 526 can transmit torqueing force to rotate the tissue removal element 166 even when the drive shaft 520 is bent.

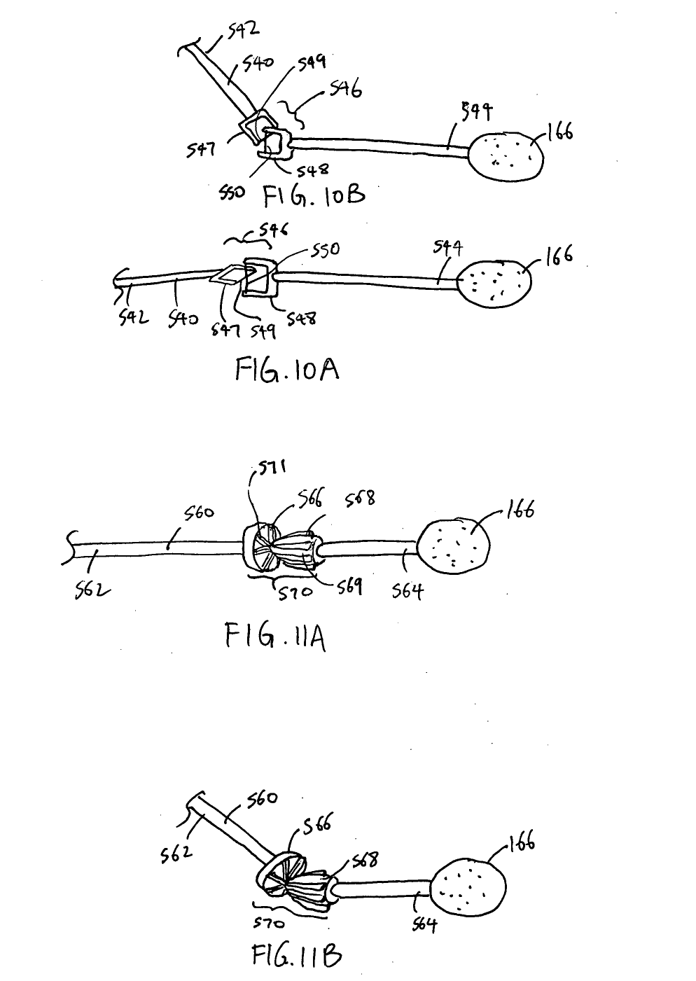

FIGS. 10A and 10B illustrate another drive shaft 540 that can be used with any of the embodiments of the tissue removal probe described herein. The drive shaft 540 includes a proximal portion 542, a distal portion 544, and a U-joint 546 connected between the proximal and the distal portions 542, 544. The U-joint 546 includes a first U-shape connector 547 secured to the proximal portion 542, a second U-shape connector 548 secured to the distal portion 544, a first shaft 549, and a second shaft 550. The second shaft 550 has an opening (not shown) which allows the first shaft 549 to extend through and to rotate. The proximal and the distal portions 542, 544 can be made from a relatively stiff materials such that the proximal and the distal portions 542, 544 remain substantially unbent during use. In such cases, a bending of the drive shaft 540 takes place at the U-joint 546, which allows the distal portion 544 to bent relative to the proximal portion 542. The U-joint 546 can transmit torqueing force to rotate the tissue removal element 166 even when the drive shaft 540 is bent. As shown in FIG. 10B, because the first shaft 549 is rotatable relative to the second shaft 550, the proximal portion 540 can be rotated (or bent) relative to the distal portion 544. In such configuration, torqueing force can be transmitted from the proximal portion 542 to the distal portion 544 via the first and the second shafts 549, 550.

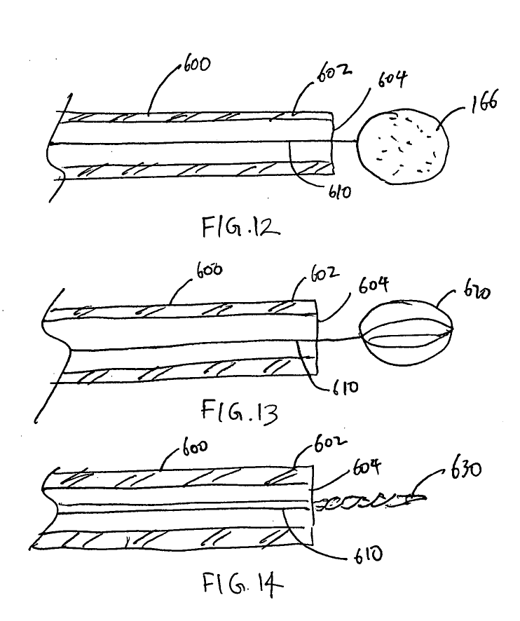

FIGS. 11A and 11B illustrate another drive shaft 560 that can be used with any of the embodiments of the tissue removal probe described herein. The drive shaft 560 includes a proximal portion 562, a distal portion 564, and a bevel gear 570. The bevel gear 570 includes a first gear 566 mounted on a distal end of the proximal portion 562, and a second gear 568 mounted on a proximal end of the distal portion 564. The second gear 568 has deep grooves 569 for engaging with teeth 571 of the first gear 566. Such configuration allows the second gear 568 to engage the first gear 566 when the distal portion 564 is rotated at an angle relative to the proximal portion 562. The proximal and the distal portions 562, 564 can be made from a relatively stiff materials such that the proximal and the distal portions 562, 564 remain substantially unbent during use. In such cases, a bending of the drive shaft 560 takes place at the bevel gear 570, which allows the distal portion 564 to bent relative to the proximal portion 562. The bevel gear 570 can transmit torqueing force to rotate the tissue removal element 166 even when the drive shaft 560 is bent (FIG. 11B). Particularly, when the drive shaft 560 is in its bent configuration, the teeth 571 first gear 566 engages with the second gear 568 at different location along the grooves 569, thereby allowing the torqueing force be transmitted from the proximal portion 562 to the distal portion 564.

Although the tissue removal probe has been described as having the cutting window 130, in alternative embodiments, the cutting window 130 is optional, and the tissue removal probe does not include the cutting window 130. FIG. 12 illustrates a distal portion of a probe shaft 600 that can be employed with any of the embodiments of tissue removal probe described herein. The probe shaft 600 has a distal end 602 and a distal tip opening 604, which allows a drive shaft 610 to extend through the probe shaft 600. The drive shaft 610, which can be any of the drive shafts described herein, is secured to the tissue removal element 166. In the illustrated embodiments, the probe shaft 600 allows the tissue removal element 166 to be completely exposed, such that the tissue removal element 166 cuts and abrades tissue on all sides of the probe shaft 600.

Although the tissue removal element 166 has been described as a burr, the scope of the invention should not be so limited. Alternatively, the tissue removal element 166 can have a variety of shapes, sizes, and configurations, so long as the tissue removal element is capable of cutting, deforming, and/or abrading a target bone tissue. In some embodiments, a cutting basket 620 (FIG. 13) can be used as the tissue removal element. In such cases, the cutting basket 620 can be made from filaments having sharp edges, thereby providing bone cutting/drilling capability. In other embodiments, the cutting basket 620 includes abrasive particles, such as diamond dust, disposed on surfaces of the filaments, for cutting, digging, and/or sanding against target bone tissue. In some embodiments, the cutting basket 620 can be made from a resiliently elastic metal, such as nitinol, which allows the cutting basket 620 to be stretched into a low profile when resided within the lumen of the probe shaft 600, and allows the cutting basket 620 to expand when outside the lumen of the probe shaft 600.

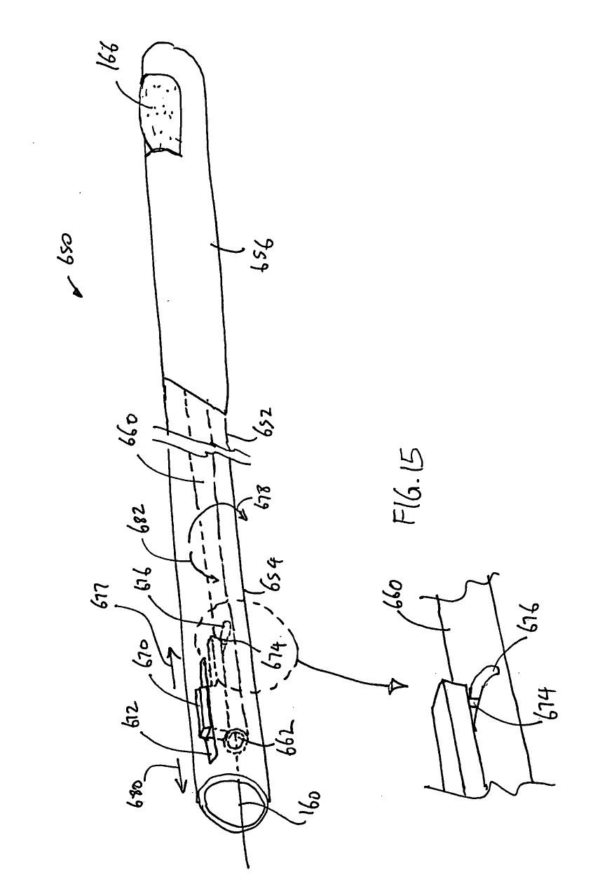

In other embodiments, the tissue removal element can be a drill bit 630 (FIG. 14). The drill bit 630 can be used to drill a hole or a channel in bone tissue.

Any of the embodiments of the tissue removal probes described herein can further include an actuator for positioning the distal portion of the probe. For example, FIG. 15 illustrates a tissue removal probe 650 in accordance with a preferred embodiment of the invention. The tissue removal probe 650 includes a probe shaft 652 having a proximal probe shaft portion 654 and a distal probe shaft portion 656 that is rotatably coupled to the proximal portion 654. A shaft 660 is secured to the distal portion 656 for positioning the distal portion 656 relative to the proximal portion 654. The shaft 160 extends within a lumen 662 of the shaft 660 and connects to the tissue removal element 166 at a distal end of the probe 650. The probe 650 includes a switch 670 for positioning the distal portion 656 relative to the proximal portion 654 of the probe shaft 652. The switch 670 includes a pin 674, and is slidable within an opening 672 at a wall of the proximal portion 654. The pin 674 is positioned within an oblique slot 676 (e.g., a slot having an axis that forms an angle with a longitudinal axis of the shaft 660) at the shaft 660, and is configured to rotate the shaft 660 in response to a positioning of the switch 670. Particularly, distal advancement of the switch 670 in a first direction 677 will cause the shaft 660 to rotate in a first direction 682, and proximal retraction of the switch 670 in a second direction 680 will cause the shaft 660 to rotate in a second direction 678. It should be noted that other types of switch can also be used to position the distal portion 656. For examples, in other embodiments, the tissue removal probe 650 can include an electrical switch coupled to a motor, or other types of mechanical switches, for rotating the shaft 660.

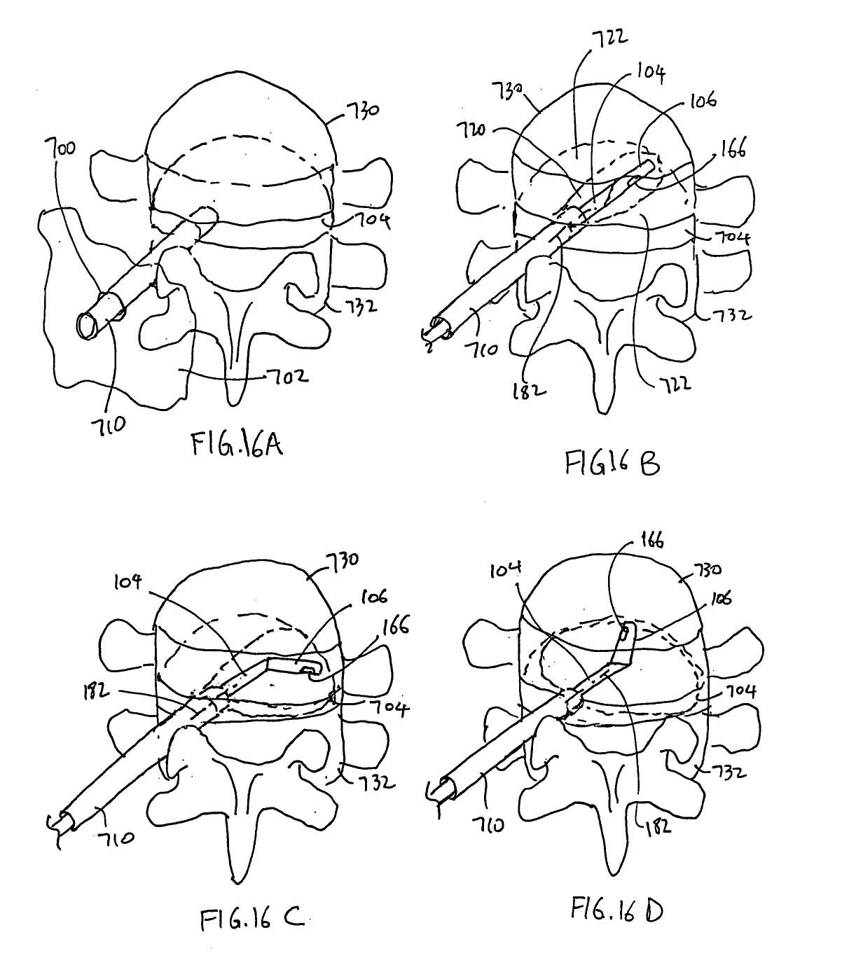

Having described the structure of various embodiments of a tissue removal probe, its operation will now be described with reference to FIGS. 16A-16C, in removing tissue from an anatomical body. Particularly, a method of performing a discectomy on a herniated intervertebral disc will now be described with reference to the tissue removal probe 100 of FIGS. 5A and 5B. It should be noted, however, that other tissue, such as the cancellous tissue within a vertebral body, can also be removed by the tissue removal probe 100. In addition, a similar method can also be employed for other embodiments of the tissue removal probe described herein.

First, a cannula 710 is introduced through a small incision 700 in the back 702 of a patient and into a herniated disc 704 situated between a top vertebra 730 and a bottom vertebra 732 (FIG. 16A). In some circumstances, a laminectomy may have to be performed to access the disc 704. In such cases, the cannula 710 may be used to bore through the lamina (not shown). Torsional and/or axial motion may be applied to the cannula 710 to facilitate boring of the lamina. The torsional and/or axial motion may be applied manually or mechanically (i.e., by a machine). An object, such as a hammer or a plunger, may also be used to tap against the cannula 710 in order to facilitate boring through the lamina. Alternatively, a stylet (not shown) can be introduced through the cannula lumen (not shown in FIG. 16A) to create a passage through the lamina. In other embodiments, a separate drill or bone cutting device can be used to bore or cut a passage through the lamina prior to placement of the cannula 710.

Next, the tissue removal probe 100 is introduced through the cannula lumen until the distal portion 106 of the probe shaft 102 is at least partially out of the cannula lumen (FIG. 16B). The tissue removal probe 100 can either be introduced into the cannula lumen prior to introduction of the cannula 710 into the patient's back (in which case, the tissue removal probe 100 will be housed within the cannula lumen during introduction of the cannula 710) or can be introduced into the cannula lumen after the cannula 710 has been introduced into, and properly positioned, within the disc 704.

Next, the driver 168 is activated to rotate the tissue removal element 166, which cuts and/or abrades disc tissue with which it comes in contact. The proximal shaft portion 104 can be advanced distally or retracted proximally to position the distal end 106 axially. The proximal shaft portion 104 can also be rotated about the longitudinal axis 182 to face the cutting window 130 in a different radial position such that the tissue removal element 166 can cut and/or abrade different tissue at the disc 704. Depending on a size of the cannula lumen, the tissue removal probe 100 can also be positioned (e.g., tilted or translated) within the confinement of the cannula lumen to place the tissue removal element 166 at desired positions. It should be noted that, during the tissue removal procedure, the removed tissue can be aspirated from the herniated disc 704 using an aspirator. Aspiration of the tissue can be accomplished via the cannula or through another cannula. Alternatively, as previously described, aspiration can be accomplished via the tissue removal probe 100 itself if the irrigation tube and the aspiration tube are provided.

Due to the confinement by the cannula lumen, the tissue removal probe 100 only removes a portion 720 of the disc 704 along the axis of the cannula lumen (FIG. 16B). If a remaining portion 722 of the disc 704 off-axis from the cannula lumen is desired to be removed, the handle 170 can be rotated to rotate the distal shaft portion 106 relative to the proximal shaft portion 104, such that the tissue removal probe 100 has a bent or configuration, i.e., the shaft portions 104, 106 are placed in their axially non-aligned configuration (FIG. 16C). If the probe 100 includes the switch 670, the switch 670 can be manipulated to rotate the distal shaft portion 106 relative to the proximal shaft portion 104. The driver 168 can be activated again to rotate the tissue removal element 166, which cuts and/or abrade the tissue.

If desired, the handle 170 can be rotated to bring the tissue removal probe 100 back to its rectilinear configuration. The proximal shaft portion 104 can then be rotated about the longitudinal axis 182 such that the cutting window 130 faces a different radial position. The handle 170 is then rotated again to provide the tissue removal probe 100 a bent configuration in a different direction (FIG. 16D), thereby allowing the tissue removal probe 100 to cut and/or abrade disc tissue at other locations in the disc 704. Rather than bringing the tissue removal probe 100 back to its rectilinear configuration (i.e., the shaft portions 104, 106 are placed in their axially aligned relationship), the proximal shaft portion 104, while the tissue removal probe 100 is in the bent configuration, can be positioned (e.g., advanced, retracted, rotated, tilted) to place the tissue removal element 166 in contact with different disc tissue, thereby removing additional disc tissue.

After the discectomy has been completed (i.e., the herniated disc material has been removed, or in some cases, the entire herniated disc has been removed), the cannula 710, along with the tissue removal probe 110, is removed from the patient's body. Alternatively, prior to total removal of the cannula 710, the tissue removal probe 100 can be removed, and a therapeutic media, such as a drug or disc replacement material can be delivered through the cannula lumen into the disc 704.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. In addition, an illustrated embodiment needs not have all the aspects or advantages of the invention shown. An aspect or an advantage described in conjunction with a particular embodiment of the present invention is not necessarily limited to that embodiment and can be practiced in any other embodiments of the present invention even if not so illustrated. Thus, the present invention is intended to cover alternatives, modifications, and equivalents that may fall within the spirit and scope of the present invention as defined by the claims.

1. A tissue removal probe, comprising:
a probe shaft having a proximal shaft portion and a distal shaft portion coupled together at an interface, wherein the proximal and distal shaft portions are configured to be positioned relative to each other, at the interface, between an axially aligned relationship and an axially non-aligned relationship;
a drive shaft disposed within the probe shaft; and
a tissue removal element mounted to the drive shaft.

2. The probe of claim 1, wherein the proximal and distal shaft portions are rigid.

3. The probe of claim 1, wherein the proximal and distal shaft portions are straight.

4. The probe of claim 1, wherein the proximal and distal shaft portions are rotatably coupled to each other.

5. The probe of claim 1, wherein the proximal and distal shaft portions are hingedly coupled to each other.

6. The probe of claim 1, wherein the distal shaft portion includes a window through which the tissue removal element is exposed.

7. The probe of claim 1, wherein the tissue removal element extends distally of the distal shaft portion.

8. The probe of claim 1, wherein the tissue removal element is rotatable.

9. The probe of claim 1, wherein the drive shaft is flexible.

10. The probe of claim 1, wherein the drive shaft has a proximal rigid shaft portion associated with the proximal probe shaft portion, a distal rigid shaft portion associated with the distal probe shaft portion, and a linkage coupling the proximal and distal drive shaft portions.

11. The probe of claim 1, further comprising an adapter mounted to the proximal shaft portion, the adapter configured for mating with a drive unit.

12. A tissue removal probe, comprising:
a probe shaft having a proximal shaft portion with a first beveled end, and a distal shaft portion with a second beveled end rotatably engaged with the first beveled end, whereby an angle between the shaft portions may be varied by rotating the shaft portions relative to each other;
a drive shaft disposed within the probe shaft; and
a tissue removal element mounted to the drive shaft.

13. The probe of claim 12, wherein the proximal and distal shaft portions are rigid.

14. The probe of claim 12, wherein the proximal and distal shaft portions are straight.

15. The probe of claim 12, wherein the first and second beveled ends are beveled at the same angle.

16. The probe of claim 12, wherein the tissue removal element is rotatable.

17. The probe of claim 12, wherein the drive shaft is flexible.

18. The probe of claim 12, wherein the drive shaft has a proximal rigid shaft portion associated with the proximal probe shaft portion, a distal rigid shaft portion associated with the distal probe shaft portion, and a linkage coupling the proximal and distal drive shaft portions.

19. The probe of claim 12, further comprising an adapter mounted to the proximal shaft portion, the adapter configured for mating with a drive unit.

20. The probe of claim 12, further comprising a rod rotatably disposed through the proximal shaft portion and fixedly coupled to the distal shaft portion.

21. The probe of claim 20, further comprising a deformable connector coupled between the rod and the distal shaft portion adjacent an interface between the proximal and distal shaft portions.

22. A tissue removal probe, comprising:
a probe shaft having proximal and distal shaft portions;
a hinge coupled between the proximal and distal shaft portions, whereby an angle between the shaft portions may be varied by hinging the shaft portions relative to each other;
a drive shaft disposed within the probe shaft; and
a tissue removal element mounted to the drive shaft.

23. The probe of claim 22, wherein the proximal and distal shaft portions are rigid.

24. The probe of claim 22, wherein the proximal and distal shaft portions are straight.

25. The probe of claim 22, wherein the tissue removal element is rotatable.

26. The probe of claim 22, wherein the drive shaft is flexible.

27. The probe of claim 22, wherein the drive shaft has a proximal rigid shaft portion associated with the proximal probe shaft portion, a distal rigid shaft portion associated with the distal probe shaft portion, and a linkage coupling the proximal and distal drive shaft portions.

28. The probe of claim 22, further comprising an adapter mounted to the proximal shaft portion, the adapter configured for mating with a drive unit.

29. The probe of claim 22, further comprising at least one pull wire disposed through the proximal shaft portion and fixedly coupled to the distal shaft portion.

30. The probe of claim 29, wherein the hinge comprises a pin mounted to the distal shaft portion, and the at least one pull wire comprises a pair of pull wires counterwound around the pin.

31. A method of removing tissue from a patient using a probe having a probe shaft with proximal and distal shaft portions and a tissue removal element associated with the distal shaft portion, the method comprising:
introducing the probe into the patient while the proximal and distal shaft portions are in an axially aligned relationship;
placing the proximal and distal shaft portions in an axially non-aligned relationship; and
moving the tissue removal element to remove the tissue while the proximal and distal shaft portions are in the axially non-aligned relationship.

32. The method of claim 31, wherein the probe is introduced into the patient along a straight path, and the tissue is off-axis relative to straight path.

33. The method of claim 31, wherein the tissue is bone tissue.

34. The method of claim 31, wherein the tissue is intervertebral disc tissue.

35. The method of claim 31, wherein the tissue removal element is rotated to remove the tissue.

36. A medical probe, comprising:
a probe shaft having a proximal and distal shaft portions configured to rotate relative to each other;
an operative element associated with the distal shaft portion;
a rod disposed through the proximal shaft portion and fixedly coupled to the distal shaft portion, the rod comprising an obliquely extending slot; and
an actuator mounted to the proximal shaft portion in an axially sliding relationship, the actuator comprising a pin slidably engaged within the slot, whereby axial movement of the actuator rotates the distal shaft portion via the rod.

37. The probe of claim 36, whereby reciprocatable axial movement of the actuator rotates the distal shaft portion relative to the proximal shaft portion.

38. The probe of claim 36, wherein the operative element is a tissue removal element.

39. The probe of claim 36, wherein the proximal and distal shaft portions are rigid.

40. The probe of claim 36, wherein the proximal and distal shaft portions are straight.

41. The probe of claim 36, further comprising a drive shaft extending through the rod and coupled to the operative element.

42. A medical probe, comprising:
a probe shaft having proximal and distal rigid shaft portions;
a drive shaft rotatably disposed within the probe shaft, the drive shaft having a proximal rigid shaft portion associated with the proximal probe shaft portion, a distal rigid shaft portion associated with the distal probe shaft portion, and a linkage coupling the proximal and distal drive shaft portions; and
an operative element mounted to the distal drive shaft portion.

43. The probe of claim 42, further comprising an adapter mounted to the proximal probe shaft portion, the adapter configured for mating with a drive unit.

44. The probe of claim 42, wherein the linkage comprises a bellow.

45. The probe of claim 42, wherein the linkage comprises a coil.

46. The probe of claim 42, wherein the linkage comprises a U-joint.

47. The probe of claim 42, wherein the linkage comprises a beveled gear set.

48. The probe of claim 42, wherein the operative element is a tissue removal element.

49. A medical probe, comprising:
a probe shaft having a proximal shaft portion and a distal shaft portion coupled together at an interface, wherein the proximal and distal shaft portions are configured to be positioned relative to each other, at the interface, between an axially aligned relationship and an axially non-aligned relationship; and
an operative element associated with the distal shaft portion.

50. The probe of claim 49, wherein the proximal and distal shaft portions are rigid.

51. The probe of claim 49, wherein the proximal and distal shaft portions are straight.

52. The probe of claim 49, further comprising a drive shaft disposed within the probe shaft, wherein the operative element is mounted to the drive shaft.

53. The probe of claim 50, wherein the drive shaft is flexible along its length.

54. The probe of claim 50, wherein the drive shaft has a proximal rigid shaft portion associated with the proximal probe shaft portion, a distal rigid shaft portion associated with the distal probe shaft portion, and a linkage coupling the proximal and distal drive shaft portions.

55. The probe of claim 50, further comprising an adapter mounted to the proximal shaft portion, the adapter configured for mating with a drive unit.

56. A medical probe, comprising:
a probe shaft having a proximal shaft portion with a first beveled end, and a distal shaft portion with second beveled end rotatably engaged with the first beveled end, whereby an angle between the shaft portions may be varied by rotating the shaft portions relative to each other; and
an operative element associated with the distal shaft portion.

57. The probe of claim 56, wherein the proximal and distal shaft portions are rigid.

58. The probe of claim 56, wherein the proximal and distal shaft portions are straight.

59. The probe of claim 56, wherein the first and second beveled ends are beveled at the same angle.

60. The probe of claim 56, further comprising a drive shaft disposed within the probe shaft, wherein the operative element is mounted to the drive shaft.

61. The probe of claim 60, wherein the drive shaft is flexible along its length.

62. The probe of claim 60, wherein the drive shaft has a proximal rigid shaft portion associated with the proximal probe shaft portion, a distal rigid shaft portion associated with the distal probe shaft portion, and a linkage coupling the proximal and distal drive shaft portions.

63. The probe of claim 60, further comprising an adapter mounted to the proximal shaft portion, the adapter configured for mating with a drive unit.

64. The probe of claim 56, further comprising a rod rotatably disposed through the proximal shaft portion and fixedly coupled to the distal shaft portion.

65. The probe of claim 56, further comprising a deformable connector coupled between the rod and the distal shaft portion adjacent an interface between the proximal and distal shaft portions.

66. A medical probe, comprising:
a probe shaft having proximal and distal shaft portions;
a hinge coupled between the proximal and distal shaft portions, whereby an angle between the shaft portions may be varied by hinging the shaft portions relative to each other; and
an operative element associated with the distal shaft portion.

67. The probe of claim 66, wherein the proximal and distal shaft portions are rigid.

68. The probe of claim 66, wherein the proximal and distal shaft portions are straight.

69. The probe of claim 66, further comprising a drive shaft disposed within the probe shaft, wherein the operative element is mounted to the drive shaft.

70. The probe of claim 69, wherein the drive shaft is flexible along its length.

71. The probe of claim 69, wherein the drive shaft has a proximal rigid shaft portion associated with the proximal probe shaft portion, a distal rigid shaft portion associated with the distal probe shaft portion, and a linkage coupling the proximal and distal drive shaft portions.

72. The probe of claim 69, further comprising an adapter mounted to the proximal shaft portion, the adapter configured for mating with a drive unit.

73. The probe of claim 66, further comprising at least one pull wire disposed through the proximal shaft portion and fixedly coupled to the distal shaft portion.

74. The probe of claim 73, wherein the hinge comprises a pin mounted to the distal shaft portion, and the at least one pull wire comprises a pair of pull wires counterwound around the pin.

75. A method of performing a medical procedure on a patient using a probe having a probe shaft with proximal and distal shaft portions and an operative element associated with the distal shaft portion, comprising:
introducing the probe into the patient while the proximal and distal shaft portions are in an axially aligned relationship;
placing the proximal and distal shaft portions in an axially non-aligned relationship; and
operating the operative element to perform the medical procedure on the tissue while the proximal and distal shaft portions are in the axially non-aligned relationship.

76. The method of claim 75, wherein the probe is introduced into the patient along a straight path, and the tissue is off-axis relative to straight path.
